(19) United States
(12) Patent Application Publication
(10) Pub. No.: US 2024/0058312 A1
(43) Pub. Date: Feb. 22, 2024

(54) METHODS AND MATERIALS FOR TREATING BONE LOSS

(71) Applicant: The Penn State Research Foundation, University Park, PA (US)

(72) Inventors: Jagadeeshaprasad Mashanipalya Guddadarangaiah, University Park, PA (US); Prem Kumar Govindappa, University Park, PA (US); John Elfar, University Park, PA (US)

(21) Appl. No.: 18/270,969

(22) PCT Filed: Jan. 5, 2022
(86) PCT No.: PCT/US2022/011262

§ 371 Date: Jul. 5, 2023

Related U.S. Application Data
(60) Provisional application No. 63/134,414, filed on Jan. 6, 2021.

(51) Int. Cl. A61K 31/4409; A61P 19/08

(52) U.S. Cl. CPC A61K 31/4409; A61P 19/08; A61K 9/0019

(57) ABSTRACT

This document relates to methods and materials for treating bone loss. For example, compositions containing 4-aminopy-ridine (4-AP) and/or one or more derivatives of 4-AP can be administered (e.g., systemically administered) to a mammal having bone loss to treat the mammal.

### CROSS-REFERENCE To RELATED APPLICATIONS

This application claims the benefit of U.S. patent application Ser. No. 63/134,414, filed on Jan. 6, 2021. The disclosure of the prior application is considered part of (and is incorporated by reference in) the disclosure of this application.

### BACKGROUND

### 1. Technical Field

This document relates to methods and materials for treating bone loss. For example, compositions containing 4-aminopyridine (4-AP) and/or one or more derivatives of 4-AP can be administered (e.g., systemically administered) to a mammal having bone loss to treat the mammal.

### 2. Background Information

An injury to bone, such as metabolic injury or traumatic injury, can represent a risk for bone loss. For example, neuromuscular injury is typically accompanied by debilitating symptoms of neuropathic pain, impaired sensory-motor function, skeletal muscle atrophy, and bone loss. Nerve fibers innervate the trabecular bone and periosteum, and are involved in bone metabolism, osteogenic differentiation of precursor cells (osteogenesis), bone mineralization, and bone remodeling (Grässel, Arthritis Res. & Ther., 16:485 (2014); Garcia-Castellano et al., Iowa Orthopaed. J., 20:49-58 (2000); Chenu, J. Musculoskel. & Neur. Interactions, 4:132-134 (2004); and Togari et al., Jap. Dental Sci. Rev., 48:61-70 (2012)).

### SUMMARY

This document provides methods and materials for treating bone loss. For example, compositions containing 4-AP and/or one or more derivatives of 4-AP can be administered (e.g., systemically administered) to a mammal (e.g., a human) having bone loss to treat the mammal. As demonstrated herein, 4-AP can improve bone quality and can reduce the rate of or prevent bone loss. Having the ability to improve bone quality and/or reduce the rate of or prevent bone loss as described herein (e.g., by administering a composition containing 4-AP) provides a unique and unrealized opportunity to treat mammals (e.g., humans) having or experiencing bone loss. For example, the ability to prevent reduction in bone mass can improve the health of patients with nerve damage.

In general, one aspect of this document features methods for treating bone loss. The methods can include, or consist essentially of, (a) identifying a mammal as having bone loss, and (b) administering a composition including 4-AP or one or more derivatives of 4-AP to the mammal. The mammal can be a human. The administering can include a systemic administration. The systemic administration can be an intraperitoneal injection. The composition can be effective to deliver about 0.05 mg/kg to about 1 mg/kg of the 4-AP or the one or more derivatives of 4-AP to the mammal. The mammal can have a disease, disorder, or condition associated with the bone loss. The disease, disorder, or condition associated with the bone loss can be osteoporosis, a bone cancer, microtrauma associated with activity or metabolic derangement, bone pain, myalgia, cachexia, wasting, human immunodeficiency virus (HIV) infection, infectious wasting, loss of function from gravity loss and weightlessness (space bone loss), loss of function from extended recumbent positioning, or excessive radiation exposure. The mammal can be a mammal lacking a current bone fracture.

In another aspect, this document features methods for improving a microarchitecture of a bone. The methods can include, or consist essentially of, administering a composition including 4-AP or one or more derivatives of 4-AP to a mammal in need of improved bone microarchitecture. The microarchitecture can include a bone volume fraction, and the method can be effective to increase the bone volume fraction. The microarchitecture can include a trabecular thickness, and the method can be effective to increase the trabecular thickness. The microarchitecture can include a trabecular separation, and the method can be effective to decrease the trabecular separation. The microarchitecture can include a structural model index, and the method can be effective to decrease the structural model index. The mammal can be a human. The administering can include a systemic administration. The systemic administration can be an intraperitoneal injection. The composition can be effective to deliver about 0.05 mg/kg to about 1 mg/kg of the 4-AP or the one or more derivatives of 4-AP to the mammal. The mammal can be a mammal lacking a current bone fracture.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. 4-AP protects against tibial bone loss following combined nerve and muscle crush injury. The systemic dosage of 4-AP (40 μg/mouse/day/IP) for 21 days significantly improves tibial bone volume (FIG. 1A), bone mineral density (BMD; FIG. 1B), and bone mineral content (BMC; FIG. 1C). Data are expressed as the mean±SEM, *P<0.05 vs. saline group, n=6 per group.

FIGS. 2A-2B. 4-AP improves the microarchitecture of tibial bone following combined nerve and muscle crush injury. The systemic dosage of 4-AP (40 μg/mouse/day/IP) for 21 days improves the microarchitecture of tibial metaphysis (FIG. 2A) by upregulating bone volume fraction and trabecular thickness (FIG. 2B, top graphs), and also by downregulating the trabecular separation and structural model index (FIG. 2B, bottom graphs). Data are expressed as the mean±SEM, n=4 per group.

### DETAILED DESCRIPTION

This document provides methods and materials for treating bone loss. For example, compositions containing 4-AP and/or one or more derivatives of 4-AP can be administered (e.g., systemically administered) to a mammal (e.g., a human) having bone loss (e.g., nerve injury-induced bone loss and/or muscle injury-induced bone loss) to treat the mammal.

In some cases, a composition described herein (e.g., a composition containing 4-AP and/or one or more derivatives of 4-AP) can be administered to a mammal (e.g., a human) in need thereof (e.g., a human having bone loss or a human identified as being likely to experience bone loss) to reduce the rate of or prevent bone loss within the mammal. For example, a composition described herein (e.g., a composition containing 4-AP and/or one or more derivatives of 4-AP) can be administered to a mammal having bone loss to slow bone loss within the mammal by, for example, 10, 20, 30, 40, 50, 60, 70, 80, 90, 95, or more percent. For example, a composition described herein (e.g., a composition containing 4-AP and/or one or more derivatives of 4-AP) can be administered to a mammal having bone loss to slow bone loss within the mammal by, for example, at least 1.5 fold (e.g., about 1.5 fold, about 2 fold, about 2.5 fold, about 3 fold, about 3.5 fold, about 4 fold, about 5 fold, about 6 fold, or more). In some cases, a composition described herein (e.g., a composition containing 4-AP and/or one or more derivatives of 4-AP) can be administered to a mammal having bone loss to prevent further bone loss within the mammal.

In some cases, a composition described herein (e.g., a composition containing 4-AP and/or one or more derivatives of 4-AP) can be administered to a mammal (e.g., a human) in need thereof (e.g., a human having bone loss) to increase the bone mineral density (BMD) of one or more bones within the mammal. For example, a composition described herein (e.g., a composition containing 4-AP and/or one or more derivatives of 4-AP) can be administered to a mammal having bone loss to increase the BMD of one or more bones within the mammal by, for example, 10, 20, 30, 40, 50, 60, 70, 80, 90, 95, or more percent. For example, a composition described herein (e.g., a composition containing 4-AP and/or one or more derivatives of 4-AP) can be administered to a mammal having bone loss to increase the BMD of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). Any appropriate method can be used to determine the BMD of a bone within a mammal. For example, X-rays, dual-energy X-ray absorptiometry (DEXA), computer tomography scans (e.g., microcomputed tomography (micro-CT) scans), fracture assessments (e.g., fracture assessments using bone surveys), magnetic resonance imaging (MRI), and/or ultrasound scans can be used to determine the BMD of a bone within a mammal.

In some cases, a composition described herein (e.g., a composition containing 4-AP and/or one or more derivatives of 4-AP) can be administered to a mammal (e.g., a human) in need thereof (e.g., a human having bone loss) to increase the bone mineral content (BMC) of one or more bones within the mammal. For example, a composition described herein (e.g., a composition containing 4-AP and/or one or more derivatives of 4-AP) can be administered to a mammal having bone loss to increase the BMC of one or more bones within the mammal by, for example, 10, 20, 30, 40, 50, 60, 70, 80, 90, 95, or more percent. For example, a composition described herein (e.g., a composition containing 4-AP and/or one or more derivatives of 4-AP) can be administered to a mammal having bone loss to increase the BMC of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). Any appropriate method can be used to determine the BMC of a bone within a mammal. For example, X-rays, DEXA, CT scans (e.g., micro-CT scans), fracture assessments (e.g., fracture assessments using bone surveys), MRI, and/or ultrasound scans can be used to determine the BMC of a bone within a mammal.

In some cases, a composition described herein (e.g., a composition containing 4-AP and/or one or more derivatives of 4-AP) can be administered to a mammal (e.g., a human) in need thereof (e.g., a human having bone loss) to improve the microarchitecture of one or more bones within the mammal. For example, a composition described herein (e.g., a composition containing 4-AP and/or one or more derivatives of 4-AP) can be administered to a mammal having bone loss to increase the bone volume fraction of one or more bones within the mammal by, for example, 10, 20, 30, 40, 50, 60, 70, 80, 90, 95, or more percent. In some cases, a composition described herein (e.g., a composition containing 4-AP and/or one or more derivatives of 4-AP) can be administered to a mammal having bone loss to increase the bone volume fraction of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). For example, a composition described herein (e.g., a composition containing 4-AP and/or one or more derivatives of 4-AP) can be administered to a mammal having bone loss to increase the trabecular thickness of one or more bones within the mammal by, for example, 10, 20, 30, 40, 50, 60, 70, 80, 90, 95, or more percent. In some cases, a composition described herein (e.g., a composition containing 4-AP and/or one or more derivatives of 4-AP) can be administered to a mammal having bone loss to increase the trabecular thickness of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). For example, a composition described herein (e.g., a composition containing 4-AP and/or one or more derivatives of 4-AP) can be administered to a mammal having bone loss to decrease the trabecular separation of one or more bones within the mammal by, for example, 10, 20, 30, 40, 50, 60, 70, 80, 90, 95, or more percent. In some cases, a composition described herein (e.g., a composition containing 4-AP and/or one or more derivatives of 4-AP) can be administered to a mammal having bone loss to decrease the trabecular separation of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). For example, a composition described herein (e.g., a composition containing 4-AP and/or one or more derivatives of 4-AP) can be administered to a mammal having bone loss to decrease the structural model index of one or more bones within the mammal by, for example, 10, 20, 30, 40, 50, 60, 70, 80, 90, 95, or more percent. In some cases, a composition described herein (e.g., a composition containing 4-AP and/or one or more derivatives of 4-AP) can be administered to a mammal having bone loss to decrease the structural model index of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). Any appropriate method can be used to determine the microarchitecture of a bone within a mammal. For example, X-rays, DEXA, CT scans (e.g., micro-CT scans), fracture assessments (e.g., fracture assessments using bone surveys), MRI, and/or ultrasound scans can be used to determine the microarchitecture of a bone within a mammal.

Any appropriate mammal having bone loss or identified as being likely to experience bone loss can be treated as described herein (e.g., by administering a composition containing 4-AP and/or one or more derivatives of 4-AP). Examples of mammals that can have bone loss (or identified as being likely to experience bone loss) and that can be treated as described herein include, without limitation, humans, non-human primates such as monkeys, horses, bovine species, porcine species, dogs, cats, mice, rats, rabbits, and goats. In some cases, a mammal (e.g., a non-human mammal) having bone loss (or identified as being likely to experience bone loss) can be a genetically modified mammal (e.g., can be genetically modified to have, or to be likely to experience, bone loss). In some cases, a human having bone loss (or identified as being likely to experience bone loss) can be treated as described herein.

In some cases, a mammal to be treated as described herein (e.g., by administering a composition containing 4-AP and/or one or more derivatives of 4-AP) can have bone loss in one or more injured bones (e.g., can have injury induced bone loss). For example, a mammal having nerve injury-induced bone loss can be treated by administering a composition containing 4-AP and/or one or more derivatives of 4-AP. For example, a mammal having muscle injury-induced bone loss can be treated by administering a composition containing 4-AP and/or one or more derivatives of 4-AP.

In some cases, a mammal to be treated as described herein (e.g., by administering a composition containing 4-AP and/or one or more derivatives of 4-AP) can have one or more diseases, disorders, or conditions associated with bone loss. Examples of diseases, disorders, and conditions associated with bone loss include, without limitation, osteoporosis, bone cancers, bone fractures (e.g., insufficiency fractures), microtrauma (e.g., microtrauma associated with activity or metabolic derangement), bone pain, myalgia, cachexia, wasting, infections (e.g., human immunodeficiency virus (HIV) infections and infectious wasting), loss of function from gravity loss and weightlessness (space bone loss), loss of function from extended recumbent positioning, and excessive radiation exposure.

In some cases, a mammal having bone loss (or identified as being likely to experience bone loss) that lacks bone fractures can be treated as described herein (e.g., by administering a composition containing 4-AP and/or one or more derivatives of 4-AP). For example, a human with no current bone fractures who has bone loss can be treated as described herein (e.g., by administering a composition containing 4-AP and/or one or more derivatives of 4-AP).

Bone loss that can be treated as described herein (e.g., by administering a composition containing 4-AP and/or one or more derivatives of 4-AP) can affect any type of bone. In some cases, bone loss that can be treated as described herein can be in cortical bone. In some cases, bone loss that can be treated as described herein can be in cancellous bone. Examples of types of bones that can exhibit bone loss and can be treated as described herein include, without limitation, long bones, short bones, flat bones, irregular bones, sesamoid bones, spinal vertebral bones and processes, skull and skull-base bones, ear bones (e.g., stapes), and fusion mass bones (e.g., fusion masses formed after bone fusion surgery).

Bone loss that can be treated as described herein (e.g., by administering a composition containing 4-AP and/or one or more derivatives of 4-AP) can affect any bone within a mammal (e.g., a bone in any location within a mammal). In some cases, bone loss that can be treated as described herein can be in the spine of a mammal. In some cases, bone loss that can be treated as described herein can be in an arm of a mammal. In some cases, bone loss that can be treated as described herein can be in a leg of a mammal. In some cases, bone loss that can be treated as described herein can be in a hip of a mammal. In some cases, bone loss that can be treated as described herein can be in a pelvis of a mammal. In some cases, bone loss that can be treated as described herein can be in a skull of a mammal. In some cases, bone loss that can be treated as described herein can be in a hand of a mammal. In some cases, bone loss that can be treated as described herein can be in a foot of a mammal.

In some cases, methods described herein also can include identifying the mammal as having bone loss. Examples of methods for identifying a mammal as having bone loss include, without limitation, bone density tests, imaging techniques (e.g., X-rays) to determine the proportion of mineral in bones, fracture assessments (e.g., fracture assessments using bone surveys), MRI, and/or ultrasound scans. Once identified as having bone loss, the mammal can be administered or instructed to self-administer a composition described herein (e.g., a composition containing 4-AP and/or one or more derivatives of 4-AP).

A composition described herein (e.g., a composition containing 4-AP and/or one or more derivatives of 4-AP) can include 4-AP and/or any appropriate derivative(s) of 4-AP. Examples of derivatives of 4-AP that can be included in a composition described herein include, without limitation, 3,4-diaminopyridine, 3-hydroxy-4-aminopyridine, N-(4-pyridyl)-t-butyl carbamate, N-(4-pyridyl) ethyl carbamate, N-(4-pyridyl) methyl carbamate, and N-(4-pyridyl) isopropyl carbamate. In some cases, 4-AP and/or one or more derivatives of 4-AP can have a structure according to Formula I:

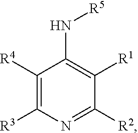

where R², R³, R⁴, and R⁵ are each independently selected from hydrogen, halogen, amine, hydroxyl, alkoxy, carboxyl, or C1-C6 alkyl. For example, R¹, R², R³, R⁴, and R⁵ can all be hydrogen. In some cases, 4-AP or a derivative thereof can be a potassium channel blocker. In some cases, 4-AP or a derivative thereof can be a calcium channel agonist. In some cases, 4-AP or a derivative thereof can be electrically active. In some cases, 4-AP or a derivative thereof can be in the form of a free base. In some cases, 4-AP or a derivative thereof can be in the form of a salt (e.g., pharmaceutically acceptable salt). When 4-AP or a derivative thereof is in the form of a salt, the salt can include any appropriate acid (e.g., an organic acid or an inorganic acid). Examples of acids that can be used to form a salt with 4-AP or a derivative thereof include, without limitation, hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, and mandelic acid.

In some cases, 4-AP and/or one or more derivatives of 4-AP can be as described elsewhere (see, e.g., U.S. Patent Application Publication No. 2018/0271847 and U.S. Pat. No. 9,993,429).

A composition described herein (e.g., a composition containing 4-AP and/or one or more derivatives of 4-AP) can include any appropriate amount of 4-AP and/or one or more derivatives of 4-AP. In some cases, a composition described herein can include from about 0.5 μM to about 10 μM (e.g., from about 0.5 μM to about 8 μM, from about 0.5 μM to about 6 μM, from about 0.5 μM to about 5 μM, from about 0.5 μM to about 3 μM, from about 0.5 μM to about 2 μM, from about 0.5 μM to about 1 μM, from about 11 μM to about 10 μM, from about 2 μM to about 10 μM, from about 4 μM to about 10 μM, from about 5 μM to about 10 μM, from about 7 μM to about 10 μM, from about 9 μM to about 10 μM, from about 1 μM to about 9 μM, from about 2 μM to about 8 μM, from about 3 μM to about 7 μM, from about 4 μM to about 6 μM, from about 1 μM to about 3 μM, from about 2 μM to about 4 μM, from about 3 μM to about 5 μM, from about 5 μM to about 7 μM, from about 6 μM to about 8 μM, or from about 7 μM to about 9 μM) of 4-AP and/or one or more derivatives of 4-AP. In some cases, a composition described herein can include from about 0.01% to about 99% (e.g., from about 0.01% to about 90%, from about 0.01% to about 80%, from about 0.01% to about 70%, from about 0.01% to about 60%, from about 0.01% to about 50%, from about 0.01% to about 40%, from about 0.01% to about 30%, from about 0.01% to about 20%, from about 0.01% to about 10%, from about 0.01% to about 5%, from about 0.01% to about 1%, from about 1% to about 99%, from about 5% to about 99%, from about 10% to about 99%, from about 20% to about 99%, from about 30% to about 99%, from about 40% to about 99%, from about 50% to about 99%, from about 60% to about 99%, from about 70% to about 99%, from about 80% to about 99%, from about 90% to about 99%, from about 10% to about 90%, from about 20% to about 80%, from about 30% to about 70%, from about 40% to about 60%, from about 10% to about 30%, from about 30% to about 50%, from about 50% to about 70%, or from about 70% to about 90%) of 4-AP and/or one or more derivatives of 4-AP.

In some cases, a composition described herein (e.g., a composition containing 4-AP and/or one or more derivatives of 4-AP) can include one or more pharmaceutically acceptable carriers (additives), excipients, and/or diluents. Examples of pharmaceutically acceptable carriers, excipients, and diluents that can be used in a composition described herein include, without limitation, saline (e.g., phosphate-buffered saline (PBS)), sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition described herein (e.g., a composition containing 4-AP and/or one or more derivatives of 4-AP) can be administered to a mammal in need thereof (e.g., a mammal having bone loss) locally or systemically. In some cases, a compositions described herein can be administered locally. For example, a composition described herein can be administered locally by injection directly into, around, and/or near an area of bone loss on a mammal (e.g., a human). In some cases, a composition described herein can be administered systemically. For example, a composition described herein can be designed for oral or parenteral (including intraperitoneal, subcutaneous, intramuscular, intravenous, and intradermal) administration to a mammal having bone loss. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition described herein can be formulated for parenteral administration (e.g., intraperitoneal injection or intravenous injection).

An effective amount (e.g., effective dose) of 4-AP and/or one or more derivatives of 4-AP in a composition described herein (e.g., a composition containing 4-AP and/or one or more derivatives of 4-AP) can vary depending on the severity of the bone loss, the route of administration, the age and general health condition of the subject, excipient usage, the possibility of co-usage with other therapeutic treatments such as use of other agents, and/or the judgment of the treating physician.

An effective amount of 4-AP and/or one or more derivatives of 4-AP in a composition described herein (e.g., a composition containing 4-AP and/or one or more derivatives of 4-AP) can be any amount that can treat bone loss on a mammal without producing significant toxicity to the mammal. An effective amount of 4-AP and/or one or more derivatives of 4-AP in a composition described herein can be any appropriate amount. In some cases, an effective amount of 4-AP and/or one or more derivatives of 4-AP in a composition described herein can be from about 0.05 milligrams per kilogram body weight (mg/kg) to about 1 mg/kg (e.g., from about 0.05 mg/kg to about 0.8 mg/kg, from about 0.05 mg/kg to about 0.6 mg/kg, from about 0.05 mg/kg to about 0.5 mg/kg, from about 0.05 mg/kg to about 0.3 mg/kg, from about 0.05 mg/kg to about 0.1 mg/kg, from about 0.1 mg/kg to about 1 mg/kg, from about 0.3 mg/kg to about 1 mg/kg, from about 0.5 mg/kg to about 1 mg/kg, from about 0.8 mg/kg to about 1 mg/kg, from about 0.1 mg/kg to about 0.9 mg/kg, from about 0.2 mg/kg to about 0.8 mg/kg, from about 0.3 mg/kg to about 0.7 mg/kg, from about 0.4 mg/kg to about 0.6 mg/kg, from about 0.1 mg/kg to about 0.3 mg/kg, from about 0.2 mg/kg to about 0.4 mg/kg, from about 0.3 mg/kg to about 0.5 mg/kg, from about 0.5 mg/kg to about 0.7 mg/kg, from about 0.6 mg/kg to about 0.8 mg/kg, or from about 0.7 mg/kg to about 0.9 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the bone loss may require an increase or decrease in the actual effective amount administered.

The frequency of administration of a composition described herein (e.g., a composition containing 4-AP and/or one or more derivatives of 4-AP) can be any frequency that can treat bone loss on a mammal without producing significant toxicity to the mammal. For example, the frequency of administration can be from about once a week to about once every two months, from about once every two weeks to about once every six weeks, or from about once every three weeks to about once a month (e.g., once every four weeks). The frequency of administration can remain constant or can be variable during the duration of treatment. In some cases, a course of treatment with a composition described herein can include rest periods. For example, a composition described herein can be administered once a month over a six-month period followed by a rest period (e.g., a one or two month rest period), and such a regimen can be repeated multiple times. As with the effective amount, various factors can influence the actual frequency of administration used for a particular application. For example, the effective amount, duration of treatment, use of multiple treatment agents, route of administration, and severity of the bone loss may require an increase or decrease in administration frequency.

An effective duration for administering a composition described herein (e.g., a composition containing 4-AP and/or one or more derivatives of 4-AP) can be any duration that treats bone loss on a mammal without producing significant toxicity to the mammal. For example, the effective duration can vary from several days to several weeks, months, or years. In some cases, the effective duration for the treatment of bone loss can range in duration from about one month to about a lifetime. Multiple factors can influence the actual effective duration used for a particular treatment. For example, an effective duration can vary with the frequency of administration, effective amount, use of multiple treatment agents, route of administration, and severity of the bone loss being treated.

In some cases, the methods and materials described herein can be used as the sole active agent used to treat a mammal (e.g., a human) having bone loss. For example, a composition containing 4-AP and/or one or more derivatives of 4-AP can be used as the sole active agent(s) used to reduce the rate of bone loss.

In some cases, methods described herein also can include administering to a mammal (e.g., a human) having bone loss one or more (e.g., one, two, three, four, five or more) additional agents used to treat bone loss in addition to a composition described herein (e.g., a composition containing 4-AP and/or one or more derivatives of 4-AP). The one or more additional agents used to treat bone loss can include any appropriate agent(s) used to treat bone loss. In some cases, an agent that can be used to treat bone loss can be a bisphosphonate. In some cases, an agent that can be used to treat bone loss can be a hormone. Examples of agents that can be used to treat bone loss include, without limitation, alendronate (e.g., BINOSTO® and FOSAMAX®), risedronate (e.g., ACTONEL® and ATELVIA®), ibandronate (e.g., BONIVA®), zoledronic acid (e.g., RECLAST® and ZOMETA®), denosumab (e.g., PROLIA® and XGEVA®, estrogen, raloxifene (e.g.,) EVISTA®, teriparatide (e.g., FORTEO®), abaloparatide (e.g., TYMLOS®), and romosozumab (EVENITY®. In cases where a mammal having bone loss is treated with a composition described herein and is treated with one or more additional agents used to treat bone loss, the additional agent(s) used to treat bone loss can be administered at the same time or independently. For example, the additional agent(s) used to treat bone loss can be formulated into a composition containing 4-AP and/or one or more derivatives of 4-AP to form a single composition. In some cases, a composition described herein can be administered first, and the one or more additional agents used to treat bone loss can be administered second, or vice versa.

The invention will be further described in the following examples, which do not limit the scope of the invention described in the claims.

### EXAMPLES

### Example 1: Bone Protection and Bone Growth with 4-Aminopyridine

This Example evaluates the effect of 4-AP against bone loss.

### Materials and Methods

### Mouse Model of Sciatic Nerve, Biceps Femoris and Quadriceps Femoris Muscle Crush Injury and Experimental Design:

Ten-week-old male C57BL/6J mice (Jackson Laboratories, Bar Harbor, Maine) weighing 25±3 g were used. Sciatic nerve crush injury was performed with muscle crush injury as described elsewhere (Govindappa et al., Int. Immunopharmacol., 82:106330 (2020)). Briefly, after intraperitoneal (IP) ketamine (100 mg/kg)/xylazine (10 mg/kg) anesthesia, the right hindlimb was shaved and prepared with alcohol swabs and povidone-iodine (Betadine). Using a stereo zoom binocular microscope, a lateral skin incision (˜3 cm) was made along the length of the femur and the sciatic nerve (SN) was bluntly exposed through the iliotibial band. Nerve crush injury was performed ˜3 mm proximal to the SN trifurcation and muscle crush injury was performed at the center of Biceps femoris (BF) and Quadriceps femoris (QF) muscle using calibrated forceps (5 mm tip width; 18-1107, Miltex Instruments) for 30 seconds (nerve) and 60 seconds (muscles), respectively. Uninjured contralateral normal limbs were kept without injury as controls. The skin was closed using surgical staples and post-operative slow-release buprenorphine (0.05 mg/kg) was given subcutaneously to all animals as an analgesic.

The experimental animals (6 animals/group) were randomized to normal saline (0.1 mL/mouse) and 4-AP (40 μg/mouse) treatment groups. 4-AP was given by IP injection immediately after surgery and daily for 21 days. Animals were euthanized using isoflurane on post-injury day 22 to harvest hindlimbs. Harvested hindlimbs were subjected to dual-energy X-ray absorptiometry (DEXA) and microcomputed tomography (micro-CT) scanning to analyze the bone quality.

### Statistical Analyses

The data were analyzed using one-way and two-way analysis of variance (ANOVA) using GraphPad Prism Version 8.2.0. All values are presented as mean±SEM. Probability (P) values of <0.05 were considered statistically significant.

### Results and Discussion

### 4-AP Anabolic Effect on the Bone

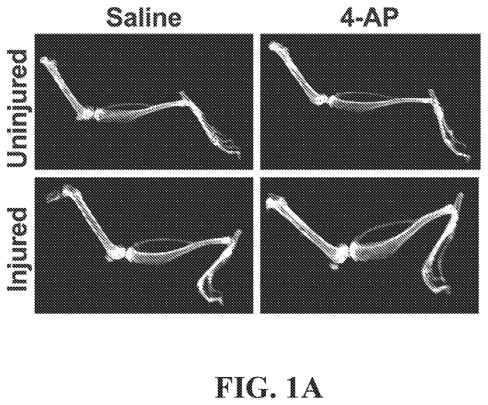

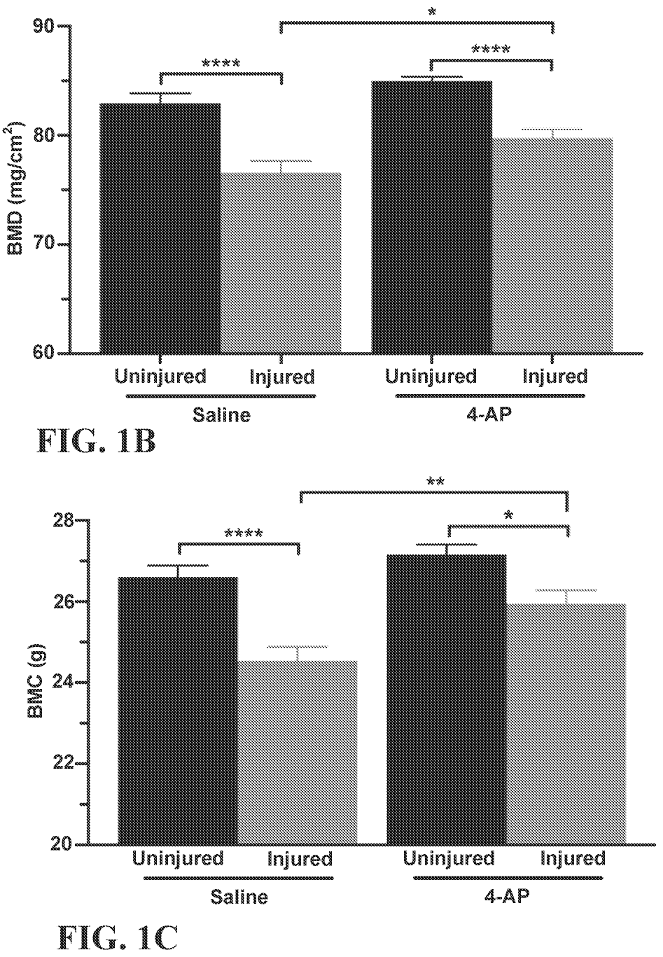

A mouse model was used to understand the anabolic effect of 4-AP on the improvement of bone quality following mangling nerve and muscle crush injury. FIG. 1A, 1B, and 1C contain representative images of tibial bone DEXA scanning, qualitative analysis of BMD (mg/cm²) and BMC (g), respectively. 4-AP treatment (vs. Saline) significantly improved BMD (79.75 mg/cm² vs. 76.57 mg/cm²) and BMC (25.94 g vs. 24.54 g) in the nerve and muscle injury associated tibial bone (FIG. 1B and 1C, *P<0.05). When compared to the contralateral tibial bone, 4-AP treatment significantly increased BMC (25.94 g vs. 27.15 g) vs. Saline treatment (24.54 g vs. 26.61 g).

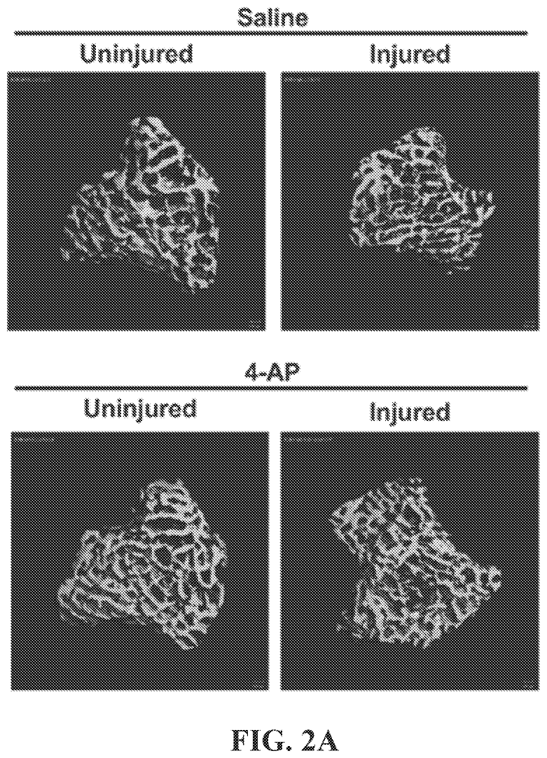

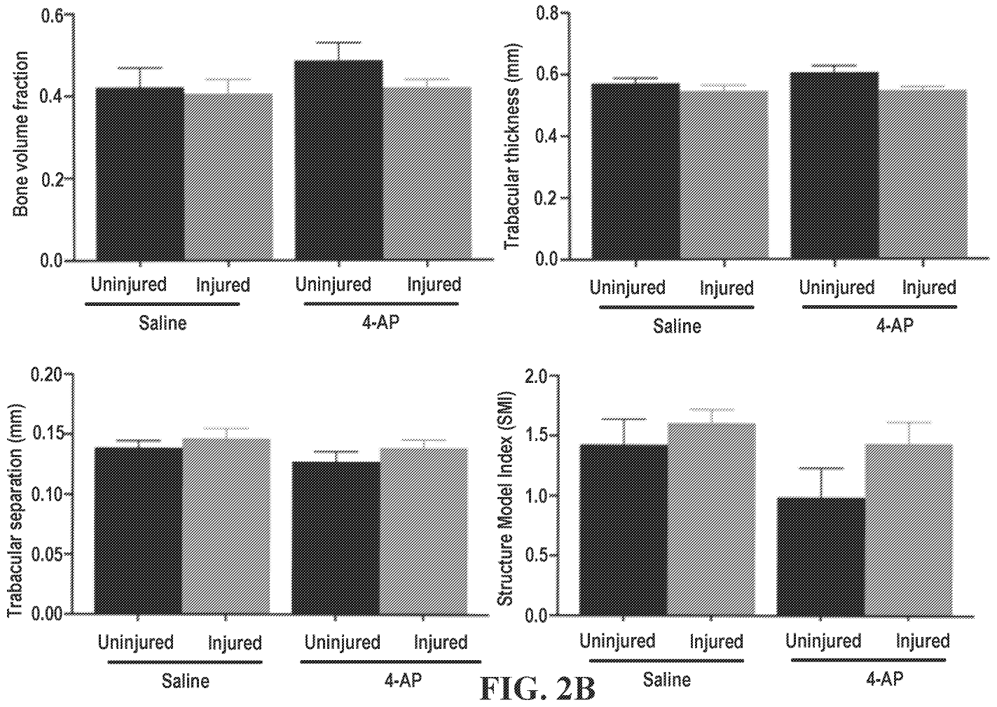

The strengthening effect of 4-AP on the tibial bone microarchitecture was examined using micro-CT. 4-AP improved tibial bone microstructural morphology by upregulating bone volume fraction and trabecular thickness (mm), and downregulated the trabecular separation (mm) and structural model index (SMI) (FIG. 2A and 2B).

Together these results demonstrate that 4-AP can be administered to a mammal to improve bone quality and/or to prevent bone loss (e.g., nerve injury-induced bone loss and/or muscle injury-induced bone loss).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating bone loss, wherein said method comprises (a) identifying a mammal as having bone loss, and (b) administering a composition comprising 4-AP or one or more derivatives of 4-AP to said mammal.

2. The method of claim 1, wherein said mammal is a human.

3. The method of claim 1, wherein said administering comprises a systemic administration.

4. The method of claim 3, wherein said systemic administration is an intraperitoneal injection.

5. The method of claim 1, wherein said composition is effective to deliver about 0.05 mg/kg to about 1 mg/kg of said 4-AP or said one or more derivatives of 4-AP to said mammal.

6. The method of claim 1, wherein said mammal has a disease, disorder, or condition associated with said bone loss.

7. The method of claim 6, wherein said disease, disorder, or condition associated with said bone loss is selected from the group consisting of osteoporosis, a bone cancer, a microtrauma associated with activity or metabolic derangement, bone pain, myalgia, cachexia, wasting, a human immunodeficiency virus (HIV) infection, infectious wasting, loss of function from gravity loss and weightlessness (space bone loss), loss of function from extended recumbent positioning, and excessive radiation exposure.

8. The method of claim 1, wherein said mammal is a mammal lacking a current bone fracture.

9. A method for improving a microarchitecture of a bone, wherein said method comprises administering a composition comprising 4-AP or one or more derivatives of 4-AP to a mammal in need of improved bone microarchitecture.

10. The method of claim 9, wherein said microarchitecture comprises a bone volume fraction, and wherein said method is effective to increase said bone volume fraction.

11. The method of claim 9, wherein said microarchitecture comprises a trabecular thickness, and wherein said method is effective to increase said trabecular thickness.

12. The method of claim 9, wherein said microarchitecture comprises a trabecular separation, and wherein said method is effective to decrease said trabecular separation.

13. The method of claim 9, wherein said microarchitecture comprises a structural model index, and wherein said method is effective to decrease said structural model index.

14. The method of claim 9, wherein said mammal is a human.

15. The method of claim 9, wherein said administering comprises a systemic administration.

16. The method of claim 15, wherein said systemic administration is an intraperitoneal injection.

17. The method of claim 9, wherein said composition is effective to deliver about 0.05 mg/kg to about 1 mg/kg of said 4-AP or said one or more derivatives of 4-AP to said mammal.

18. The method of claim 9, wherein said mammal is a mammal lacking a current bone fracture.
